(19) United States
(12) Patent Application Publication
(10) Pub. No.: US 2011/0071334 A1
(43) Pub. Date: Mar. 24, 2011

(54) FOAMING DECONTAMINATING AQUEOUS SOLUTION

(76) Inventor: Pierre Goffinet, Gif-Sir-Yvette (FR)

(21) Appl. No.: 12/994,622

(22) PCT Filed: May 26, 2009
(86) PCT No.: PCT/FR2009/050973

§ 371 Date: Nov. 24, 2010

(30) Foreign Application Priority Data
May 27, 2008 (FR) 0853437

(51) Int. Cl. A62D 3/38; A62D 3/30

(52) U.S. Cl. 588/320; 510/110

(57) ABSTRACT

The invention relates to a foaming decontaminating aqueous solution, characterized in that it comprises, by weight relative to the total weight of the composition:
0.1% to 20% of an organic or mineral water-soluble peroxidizing agent;
0.05% to 20% of an anionic surfactant;
0.1% to 20% of an amine oxide;
0 to 5% of a stabilizer for the peroxidizing agent;
0 to 10% of an amphoteric surfactant;
0 to 15% of an alkaline buffer; and
the remainder to 100% being water;
   said organic or mineral water-soluble peroxidizing agent being chosen from:
the organic peracids of general formula R′—CO₃H in which R′ is a linear or branched alkyl group containing from 1 to 4 carbon atoms and preferably from 1 to 3 carbon atoms and alkali metal, alkaline-earth metal, ammonium or hydroxylamine salts thereof;
the organic peracids of general formula HO₃C—R′—CO₃H in which R′ is a linear or branched alkyl group of 1 to 4 carbon atoms and preferably of 1 to 3 carbon atoms, and the alkali metal, alkaline-earth metal, ammonium or hydroxylamine salts thereof;
the organic peracids of general formula R″-(Φ)(CO₃H)ₙ(CO₂H)ₘ in which R″ is a hydrogen or a linear or branched alkyl group of 1 to 4 carbon atoms, (Φ) is a benzene nucleus, n=1 or 2, m=0 or 1, and the alkali metal, alkaline-earth metal, ammonium or hydroxylamine salts thereof;
the mineral peroxides chosen from H₂O₂ adducts, persalts, mineral peracids and alkali metal salts thereof,
mixtures of said peroxidizing agents;
   the pH of said aqueous solution being between 7 and 11.

The invention can be used for dismantling explosive devices and decontaminating the toxic and/or bacteriological agents contained therein.

The present invention relates to the decontamination of surfaces or the destruction of explosives liable to contain chemical or bacteriological toxic agents, in particular organophosphorus or organosulfur toxic agents and/or pollutants, especially in the field of chemical warfare agents or in the field of agriculture, for example for the decontamination of insecticides.

Many organophosphorus esters derived from phosphoric and phosphonic acids currently exist, which are used as chemical warfare agents or toxic warfare agents, such as the toxic agents or products G: “Tabun”, “Sarin” or “Soman”, or as insecticides in agriculture, for instance “paraoxon”, “diazinon” or the toxic agent VX.

These compounds have high neurotoxicity due to their phosphorylating power on cholinesterases, inhibition of which leads to death by accumulation of acetylcholine in the body.

Moreover, organosulfur toxic products also exist, such as industrial sulfides or warfare agents of the vesicant family, such as yperite (or toxic agent HD).

The existence of large stocks of warfare munitions liable to contain chemical or bacteriological toxic agents and the increasing threat of terrorist actions using explosive or dispersant systems containing such toxic agents have been the impetus for studies into effective solutions for decontamination and/or destruction.

As a general rule, the decontamination of toxic or bacteriological agents is performed by reduction, oxidation or hydrolysis.

Several types of decontaminating composition exist. Examples that may be mentioned include alkaline solutions, such as solutions of sodium hydroxide, of alkanolamines or of amines or hypochlorite solutions. These solutions have the drawback of being very corrosive toward the contaminated materials.

Other compositions containing an organic or mineral peroxidizing agent, such as a peracid or a peroxide, have been recommended, optionally in combination with a surfactant of quaternary ammonium type (FR 2 651 133 and FR 2 676 368), a sequestrant (FR 2 766 725) or a surfactant formed from an amine oxide (FR 2 766 724).

Decontaminating compositions have also been described containing chlorinated derivatives such as chloroisocyanurates (WO 00/51687 and DE 1 005 899); biocides including quaternary ammonium derivatives in combination with enzymes (WO 01/0056380) or metal hydroxide and oxide nanoparticles, especially of alkali metals, alkaline-earth metals and transition metals (U.S. Pat. No. 6,827,766).

It has been proposed to add a foaming agent to these decontaminating compositions (U.S. Pat. No. 6,376,436 and U.S. Pat. No. 2,392,936).

In many cases, it is highly desirable to make the explosive or dispersant system explode on site using a detonator or a countercharge.

In order to limit the effects of the blast of the explosion, it has already been proposed to use a foam that is capable of absorbing the pressure generated by the shock wave of the explosion (WO 94/00198). Usually, when the explosive cannot be transported, it is confined in a mobile chamber (WO 98/56465), for instance a Kevlar® tent whose inner space is entirely filled with a foam obtained in situ by diffusing a gas stream in an aqueous solution of a composition containing a foaming agent, such as a surfactant (WO 94/00198).

These foaming compositions are generally formulated based on anionic surfactants. Specifically, these anionic surfactants make it possible to obtain high swelling coefficients, excellent stability and viscoelastic behavior of the foam that are favorable to absorption of the energy of the explosion blast.

It is desirable to have available foaming compositions that are also decontaminating so as to be able simultaneously to destroy the explosive and to decontaminate the toxic agents that it is liable to contain.

Given the nature of the chemical reactions involved, for decontaminating chemical toxic agents (oxidation or nucleophilic substitution), decontaminating agents are generally used in the presence of cationic surfactants. Toxic agents are decomposed by means of a micellar catalysis reaction via microemulsions of the decontaminating agents that form in the presence of the cationic surfactant. Furthermore, the combination of cationic surfactants and of peracids has a very broad spectrum of decontaminating efficacy on biological agents (bacteria, viruses, spores and toxins).

Unfortunately, the cationic surfactants that have a high critical micellar concentration are not themselves very good foaming agents. Furthermore, their presence in foaming compositions comprising anionic surfactants leads to the formation of an electrically neutral hydrophobic complex that is very sparingly soluble in aqueous media and to destabilization of the foam.

The Applicant has now found a novel decontaminating solution free of cationic surfactants that can simultaneously reduce the blast effect of an explosive device and decontaminate the toxic and/or bacteriological agents contained therein.

Thus, the present invention relates to a foaming decontaminating aqueous solution comprising, by weight relative to the total weight of the composition:
0.1% to 20% and preferably 0.3% to 5% of an organic or mineral water-soluble peroxidizing agent as defined below;
0.05% to 20% and preferably 0.1% to 5% of an anionic surfactant;
0.1% to 20% and preferably 0.1% to 5% of an amine oxide;
0 to 5% and preferably 0.1% to 2% of a stabilizer for the peroxidizing agent;
0 to 10% and preferably 0.5% to 4% of an amphoteric surfactant;
0 to 15% and preferably 0 to 10% of an alkaline buffer;
the remainder to 100% being water;
   the pH of said aqueous solution being between 7 and 11 and preferably between 8.0 and 9.5.

The invention also relates to the composition for preparing the foaming decontaminating aqueous solution of the invention.

This composition contains all the constituents necessary for preparing the aqueous solution according to the invention, and optionally all or some of the water.

Preferably, in the composition for preparing the foaming decontaminating aqueous solution of the invention, the peroxidizing agent, and optionally its stabilizing agent, are separate from the other constituents.

Thus, preferably, said composition comprises in separate packagings I and II:
the organic or mineral water-soluble peroxidizing agent and, optionally, a stabilizer for the said water-soluble peroxidizing agent in packaging I;
the other constituents and optionally all or some of the water necessary for preparing the aqueous solution of the invention in packaging II.

Preferably, packaging II contains the constituents other than the peroxidizing agent and its stabilizer in the form of a concentrated solution, the concentration limit being the solubility limit of the constituents contained in packaging II.

Advantageously, packaging II is free of water or contains only the amount of water necessary to form a concentrated solution of the other constituents. The amount of water to be added may be readily determined by a person skilled in the art, as a function of the solubility of the other constituents.

The organic or mineral water-soluble peroxidizing agent is chosen from:
the organic peracids of general formula R′—CO₃H in which R′ is a linear or branched alkyl group containing from 1 to 4 carbon atoms and preferably from 1 to 3 carbon atoms and alkali metal, alkaline-earth metal, ammonium or hydroxylamine salts thereof;
the organic peracids of general formula HO₃C—R′—CO₃H in which R′ is a linear or branched alkylene group of 1 to 4 carbon atoms and preferably of 1 to 3 carbon atoms, and the alkali metal, alkaline-earth metal, ammonium or hydroxylamine salts thereof;
the organic peracids of general formula R″-(Φ)(CO₃H)ₙ(CO₂H)ₘ in which R″ is a hydrogen or a linear or branched alkyl group of 1 to 4 carbon atoms, (Φ) is a benzene nucleus, n=1 or 2, m=0 or 1, and the alkali metal, alkaline-earth metal, ammonium or hydroxylamine salts thereof;
the mineral peroxides chosen from H₂O₂ adducts, persalts, mineral peracids and alkali metal salts thereof, especially sodium percarbonates and persulfates such as sodium caroate (or oxone), potassium or ammonium caroate, and urea peroxide. These peroxides are optionally activated with a peroxidation activator chosen from tetraacetylethylenediamine (TAED), pentaacetylglucose (PAG), tetraacetylglucoluryl (TAGU), tetraacetylcyanuric acid (TACA), α-acetoxy-α-methyl-N,N′-(diacetyl)malonamide, acetylsalicylic acid (ASA), sodium para-acetoxybenzenesulfonate (AOBS), diacetyl dimethylglyoxime (DDG), ethylidenebenzoacetate (EBA), phthalic anhydride (PAN), benzoylimidazole (BID), sodium para-benzoxybenzenesulfonate (BOBS), sodium nonanoyloxybenzenesulfonate (NOBS) and sodium isononanoyloxybenzenesulfonate (ISONOBS);
mixtures of said peroxidizing agents.

As examples of water-soluble peroxidizing agents that are preferred for the purposes of the invention, mention may be made especially of:
peracetic acid, sodium peracetate, perpropionic acid, performic acid and potassium perpropionate;
magnesium monoperoxyphthalate hexahydrate (MPPM), perphthalic acid, perbenzoic acid, etc.;
sodium caroate, sodium percarbonate, etc.

The anionic surfactant may be chosen from the anionic agents below:
water-soluble alkylbenzenesulfonates whose alkyl groups, with a straight or branched chain, contain about 8 to 16 carbon atoms;
water-soluble alkyl ether sulfates or alkenyl ether sulfates whose alkyl or alkenyl groups, with a straight or branched chain, contain about 10 to 20 and preferably 12 to 18 carbon atoms and on average 0.5 to 8 mol and preferably 2 to 4 mol of C₂-C₄ alkylene oxide, for example ethylene oxide, propylene oxide, butylene oxide or a mixture of these alkylene oxides;
water-soluble alkyl sulfates or alkenyl sulfates whose alkyl or alkenyl groups, with a straight or branched chain, contain about 10 to 22 carbon atoms and preferably 12 to 18 carbon atoms;
water-soluble monoalkyl or dialkyl or alkenyl sulfosuccinates, whose alkyl or alkenyl groups, with a straight or branched chain, contain about 8 to 22 carbon atoms;
water-soluble linear or branched alkyl or alkenyl phosphates comprising from 8 to 22 carbon atoms;
water-soluble saturated or unsaturated fatty acid soaps comprising from 8 to 22 carbon atoms;
water-soluble α-sulfonated fatty acids comprising from 8 to 22 carbon atoms and esters thereof resulting from condensation with a saturated or unsaturated alcohol comprising from 1 to 10 carbon atoms or an alkylene oxide group resulting from the condensation of 1 to 10 ethylene or propylene oxide units;
water-soluble alkyltaurates and alkylisethionates whose alkyl chain comprises between 8 and 22 carbon atoms;
water-soluble N-alkylamide fatty acid sulfates or sulfonates comprising from 8 to 22 carbon atoms, the nitrogen atom bearing from 0 to 2 hydrogen atoms and from 0 to 2 saturated or unsaturated alkyl groups comprising from 1 to 6 carbon atoms or a group resulting from the condensation of 1 to 10 ethylene or propylene oxide units;
water-soluble alkyl benzoylsulfopropionates whose alkyl groups, with a straight or branched chain, contain from 8 to 22 carbon atoms;
water-soluble fatty acid ester sulfonates or sulfates corresponding to one of the formulae below:

R—C(═O)—O—R′—SO₃—X and R—C(═O)—O—R′—SO₄—X
in which R is a straight-chain or branched-chain alkyl group containing about 8 to 22 carbon atoms, R′ is a saturated or unsaturated alkylene group comprising from 1 to 6 carbon atoms and X is an alkali metal cation, NH₄⁺ or a cation of a C₁-C₆ alkanolamine such as monoethanolamine, diethanolamine, triethanolamine or isopropanolamine;
the water-soluble sulfonated alkyl ethers of general formula:

R—O—R′—SO₃—X
in which R is a straight-chain or branched-chain alkyl group containing about 8 to 22 carbon atoms, R′ is a saturated or unsaturated alkylene group comprising from 1 to 6 carbon atoms and X is an alkali metal cation, NH₄⁺ or a cation of a C₁-C₆ alkanolamine such as monoethanolamine, diethanolamine, triethanolamine or isopropanolamine;
mixtures of said anionic surfactants.

The above anionic surfactants are commercially available anionic agents or may be prepared via standard processes that are well known to those skilled in the art. By way of reference concerning these anionic surfactants, mention will be made of the publications below:
“Anionic surfactants” by M. Linfield (Coll. Surfactant Science Collection)—Editor Warner;
“Amphoteric Surfactants” by B. R. Bluestein et al. (Coll. Surfactant Science Collection)—Editor L. Hiltoy.

Preferably, an anionic surfactant that allows a reduction in the surface tension of water at 20° C. of at least 4.0×10⁻² N/m will be chosen.

The foaming power of the composition according to the invention is determined according to standard NF T 73404, also known as the Ross-Miles test.

The composition may contain from 0 to 10% and preferably 0.5% to 4% of an amphoteric surfactant such as (C₈-C₁₈)alkylbetaines, (C₈-C₁₈)alkylsulfobetaines and (C₈-C₁₈)alkylamphocarboxyglycinates or (C₈-C₁₈)alkylamphocarboxypropionates or mixtures thereof.

The amine oxide used in the foaming decontaminating composition according to the invention is any amine oxide that is capable of effecting micellar catalysis within the composition. The oxidation and nucleophilic substitution reactions are thus favored by increasing the surface for exchange between the foaming decontaminating composition of the invention and the toxic agent.

The function of the amine oxide within the composition according to the invention is entirely different to the stabilizing function described in GB 1 089 997.

It has been found that the use of an amine oxide as micellar catalyst instead of a cationic surfactant does not reduce, but, on the contrary, considerably reinforces the foaming power of the decontaminating composition.

Advantageously, the amine oxide will be chosen from:
amine oxides corresponding to the formulae below:

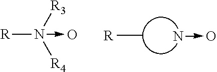

in which:
R is a linear or branched alkyl or alkenyl group containing 8 to 22 carbon atoms, optionally interrupted with a group —O—C(═O); —O—; —O—NH₂— or —O—NR₁R₂—;
R₃ and R₄, which may be identical or different, represent a C₁-C₄ alkyl or a group of formula [(CH₂)ₘ—O]ₙ—H in which m and n are between 1 and 4;

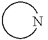

is a heterocycle containing 2 to 6 carbon atoms and at least one nitrogen atom;
   mixtures of said amine oxides.

The above amine oxides are commercially available amine oxides or may be obtained via standard processes that are well known to those skilled in the art, for example by oxidation reaction of tertiary amines with peroxidizing agents, for example hydrogen peroxide.

Examples of amine oxides that correspond to one or other of the above formulae are:
N-lauryldimethylamine oxide;
laurylpyridine oxide;
N-cetyldiethylamine oxide;
octyl-pyridine oxide;
N-myristylbis(2-hydroxyethyl)amine oxide.

Any alkaline buffer capable of adjusting the pH of the composition of the invention to between 7 and 11 and preferably between 8.0 and 9.5 may be used. The alkaline buffer is preferably chosen from alkali metal or alkaline-earth metal carbonates, bicarbonates, borates, silicates, citrates, tartrates and phosphates, or mixtures thereof.

Sodium or potassium carbonate, borate, phosphate (ortho-, pyro- or tripolyphosphate) or citrate buffers are particularly preferred, in particular the carbonates.

Furthermore, the composition of the invention may optionally comprise one or more stabilizers for the peroxidizing agent, such as:
organic phosphonate acids and salts, such as 1-hydroxyethylene-1,1-diphosphonic acid (HEDP), ethylenediaminetetramethylenephosphonate (EDTMP) or diethylenetriaminepentamethylenephosphonic acid (DTPMP);
aminopolycarbonate acids and salts, such as ethylenediaminetetraacetic acid (EDTA) and diethylenetriaminepentaacetic acid (DTPA);
citric acid and salts thereof;
8-hydroxyquinoline;
dipicolinic acid and salts thereof;
traces of colloidal silver salts;
tartaric acid and salts thereof;
sequesterants for scavenging divalent or trivalent metals.

Optionally, the aqueous solution according to the invention may also comprise a nonionic surfactant of the family of fatty alcohols and ethoxylated fatty alcohols, ethoxylated-propoxylated fatty alcohols, fatty amides, ethoxylated fatty amides, especially diethanolamides, alkylpolyglucosides, etc.

The aqueous solution according to the invention may also comprise viscosity regulators, such as xanthan, carrageenan, gellan or guar gums, Laponite® (colloidal clay: Mg, Na and Li silicate), carboxymethylcellulose, methylcellulose, hydroxyethylcellulose, hydroxypropylcellulose, modified starch, polyethylene glycol, polyacrylic acid and polyacrylates (carbomers), etc., preferably with a molecular mass of between 400 and 400 000 daltons.

Finally, the aqueous solution according to the invention may, still optionally, contain alcoholic or glycolic solvents, polyglycols, glycol ethers, hydrotropic agents (urea, ethoxylated phenol containing 3-6 mol of ethylene oxide, and alkali metal or ammonium cumenesulfonates, toluenesulfonates or xylenesulfonates).

The aqueous solutions according to the invention are obtained by extemporaneous addition of the peroxidizing agent to the aqueous solution containing the other constituents.

The invention also relates to the process for decontaminating surfaces, which consists in:
1) generating a foam from the aqueous solution according to the invention;
2) placing said foam in contact with the surface to be decontaminated for a few minutes;
3) recovering the effluents after liquefying the foam.

The invention also relates to the process for destroying explosives liable to contain chemical or bacteriological toxic agents, which consists in:
1) generating a foam from an aqueous solution according to the invention;
2) confining the explosives to be destroyed in a mobile chamber;
3) spraying the foam obtained in step 1) into said chamber;
4) igniting said explosives to be destroyed;
5) recovering the effluents after liquefying the foam.

The decontaminating efficacy and the foaming power of the aqueous solutions according to the invention on toxic warfare agents was evaluated on analogs of toxic agents, namely analogs of yperite, VX and the G products.

These analogs of toxic agents are molecules with a lower degree of toxicity, but a chemical structure and reactivity demonstrated as being equivalent to that of the actual toxic agents.

### Yperite (HD) Analog

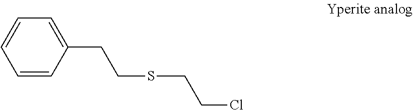

This product proposed as analog is chloroethyl phenylethyl sulfide (to be compared with yperite: ClCH₂CH₂SCH₂CH₂Cl), and characterized by:
the presence of a sulfur atom
the presence of a Cl in beta position to the sulfur atom to mimic the formation of a sulfonium intermediate.

Like yperite, it is generally destroyed by oxidation; the sulfur atom is oxidized to chlorosulfoxide and then to chlorosulfone; the vinyl sulfone forms in alkaline medium according to the reaction scheme below, in which R=phenylethyl:

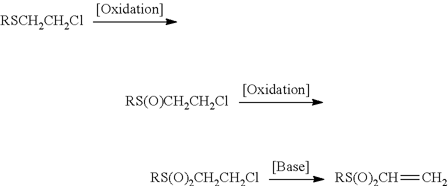

The reaction is monitored by gas/mass chromatography and thin-layer chromatography.

### VX Analog

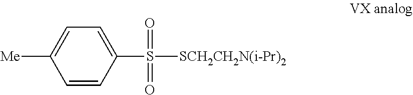

This product, like its para-fluoro homolog, is a good analog of VX: CH₃CH₂—O—P(O)(CH₃)—S—CH₂CH₂—N(iPr)₂, especially due to the presence of the β-aminothiol function. Specifically, the oxidation should take place on the sulfur and avoid the formation of an N-oxide (highly toxic).

Like VX, it is destroyed in alkaline medium or in the presence of an oxidizing acid, giving either a disulfide or a sulfonic acid.

The reaction is monitored over time by TLC (revelation with iodine).

### Analog of the G Products

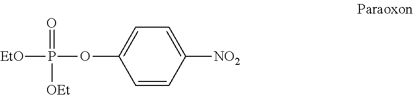

Commercial insecticide; excellent analog of G products:

These products are generally destroyed via a nucleophilic substitution reaction of the fluorine (Sarin, Soman) or of the cyano group (Tabun)

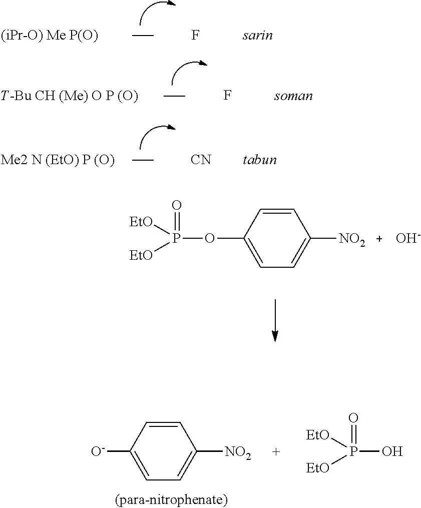

The reaction is monitored:
by gas chromatography (after extraction with ethyl acetate/hexane)

The invention will now be described in greater detail by the illustrative but non-limiting examples below.

### EXAMPLE 1

### Foaming Decontaminating Aqueous Solution

The peroxidizing agent was mixed with the other constituents and water in a container, to obtain the aqueous solution below:

| | |
|---|---|
| Peracetic acid (37%) | 2.50% |
| N-Lauryldimethylamine oxide (30%) | 3.00% |
| Na₂CO₃ | 3.50% |
| Na DTPMP (30%) | 4.00% |
| Sodium lauryl sulfate | 0.30% |
| Fatty alkyl ether laurylsulfosuccinate | 0.20% |
| Water | 83.00% |

The pH of the solution was 8.2.

### EXAMPLE 2

### Foaming Decontaminating Aqueous Solution

The peroxidizing agent was mixed with the other constituents and water in a container, to obtain the aqueous solution below:

| | |
|---|---|
| Peracetic acid (37%) | 3.00% |
| N-Laurylpyridine oxide | 2.00% |
| Sodium disilicate | 5.50% |
| Sodium HEDP (30%) | 1.00% |
| Sodium lauryl ether sulfate (28%) | 8.00% |
| Water | 80.50% |

The pH of the solution was 9.

### EXAMPLE 3

### Foaming Decontaminating Aqueous Solution

The peroxidizing agent was mixed with the other constituents and water in a container, to obtain the aqueous solution below:

| | |
|---|---|
| MPPM | 12.00% |
| N-cetyldiethylamine oxide | 7.00% |
| K₂CO₃ | 3.50% |
| Sodium citrate | 6.00% |
| Triethanolamine lauryl sulfate | 0.30% |
| Water | 68.50% |

The pH of the solution was 8.5.

### Comparative Tests

In these tests, the aqueous solutions according to the invention described in Examples 1 to 3 were compared with the standard decontaminating composition A below, to which was added the foaming composition B.

### Procedure for Measuring the Chemical Decontamination on HD and VX Analogs

5 g of the foaming decontaminating aqueous solution of the invention were deposited using a pipette onto a glass plate coated with HD or VX analogs in a Sovirel flask.

The Sovirel flask was stirred for 20 minutes and the oxidation reaction was then quenched with 2 ml of thiosulfate.

Extraction was then performed (of the analog and its metabolites remaining on the plate) with an ethyl acetate/cyclohexane mixture (50/50).

Analysis of the residual chemical agent (analog+metabolites) was performed by GC (Silicone SE column) and by thin-layer chromatography.

### Procedure for Measuring the Chemical Decontamination on G Product Analogs

5 g of the foaming decontaminating aqueous solution were deposited on the G product analog in a Sovirel flask.

The Sovirel flask was then placed on a shaker plate with maximum agitation for 20 minutes.

The following were then introduced into the Sovirel flask:
10.0 ml of internal standard solution (dibutyl sebacate) at 0.1% in an ethyl acetate/hexane mixture (50/50).

The Sovirel flask was stoppered once again and shaken manually for 10 seconds. The supernatant organic phase was recovered immediately in another 25 ml Sovirel flask containing sodium sulfate and injected in GC onto an OV1 glass column.

The results obtained are given in the Table I below:

TABLE I

| Foaming power | | | Decontamination after 60 | | |
|---|---|---|---|---|---|
| (Ross Miles test - ISO 696) | | | minutes | | |
| | | Foam | | | Product G |
| | Time (in | height | HD | | (paraoxon) |
| Solution | min) | (in ml) | analog | VX analog | analog |
| Example 1 | 0 | 470 | | | |
| | 3 | 415 | | | |
| | 5 | 400 | | | |
| | 10 | 400 | | | |
| | 20 | 400 | | | |
| | 30 | 390 | | | |
| | 60 | | 100% | 90% | 80% |
| Example 2 | 0 | 410 | | | |
| | 3 | 390 | | | |
| | 5 | 380 | | | |
| | 10 | 380 | | | |
| | 20 | 380 | | | |
| | 30 | 380 | | | |
| | 60 | | 100% | 90% | 90% |
| Example 3 | 0 | 440 | | | |
| | 3 | 415 | | | |
| | 5 | 400 | | | |
| | 10 | 390 | | | |
| | 20 | 380 | | | |
| | 30 | 370 | | | |
| | 60 | | 90% | 80% | 70% |
| Decontaminating | 0 | 320 | | | |
| solution A | 3 | 290 | | | |
| (17%) + | 5 | 270 | | | |
| foaming solution | 10 | 260 | | | |
| B (4%) + | 20 | 150 | | | |
| demineralized | 30 | 60 | | | |
| water: qs 100% | 60 | | 30% | 40% | 40% |

| | Material | Function | mass % |
|---|---|---|---|
| Standard | Magnesium | Peroxidizing agent | 47.0 |
| decontaminating | monoperoxy- | | |
| solution: A | phthalate | | |
| | hexahydrate | | |
| | (MPPM) | | |
| | Myristalkonium | Micellar catalyst | 8.0 |
| | chloride | | |
| | Na₂CO₃ | pH buffer | 18.0 |
| | NaOH | Alkalinizing agent | 3.0 |
| | Sodium gluconate | Stabilizer | 24.0 |
| Foaming | Triethanolamine | Foaming anionic | 12.0 |
| solution: B | lauryl sulfate | surfactant | |

1. A foaming decontaminating aqueous solution, characterized in that it comprises, by weight relative to the total weight of the composition:
0.1% to 20% of an organic or mineral water-soluble peroxidizing agent;
0.05% to 20% of an anionic surfactant;
0.1% to 20% of an amine oxide;
0 to 5% of a stabilizer for the peroxidizing agent;
0 to 10% of an amphoteric surfactant;
0 to 15% of an alkaline buffer; and
the remainder to 100% being water;
said organic or mineral water-soluble peroxidizing agent being chosen from:
the organic peracids of general formula R′—CO₃H in which R′ is a linear or branched alkylene group containing from 1 to 4 carbon atoms and preferably from 1 to 3 carbon atoms and alkali metal, alkaline-earth metal, ammonium or hydroxylamine salts thereof;
the organic peracids of general formula HO₃C—R′—CO₃H in which R′ is a linear or branched alkyl group of 1 to 4 carbon atoms and preferably of 1 to 3 carbon atoms, and the alkali metal, alkaline-earth metal, ammonium or hydroxylamine salts thereof;
the organic peracids of general formula R″-(Φ)(CO₃H)ₙ(CO₂H)ₘ in which R″ is a hydrogen or a linear or branched alkyl group of 1 to 4 carbon atoms, (Φ) is a benzene nucleus, n=1 or 2, m=0 or 1, and the alkali metal, alkaline-earth metal, ammonium or hydroxylamine salts thereof;
the mineral peroxides chosen from H₂O₂ adducts, persalts, mineral peracids and alkali metal salts thereof,
mixtures of said peroxidizing agents;
the pH of said aqueous solution being between 7 and 11.

2. The solution as claimed in claim 1, characterized in that the pH is between 8.0 and 9.5.

3. The solution as claimed in claim 1, characterized in that it comprises, by weight relative to the total weight of the composition:
0.3% to 5% of an organic or mineral water-soluble peroxidizing agent;
0.1% to 5% of an anionic surfactant;
0.5% to 4% of an amphoteric surfactant;
0.1% to 5% of an amine oxide;
0.1% to 2% of a stabilizer for the peroxidizing agent;
0 to 10% of an alkaline buffer; and
the remainder to 100% being water.

4. The solution as claimed in claim 1, characterized in that the amine oxide is chosen from: 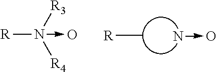
in which:
R is a linear or branched alkyl or alkenyl group containing 8 to 22 carbon atoms, optionally interrupted with a group —O—C(═O); —O—; —O—NH₂— or —O—NR₁R₂—;
R₃ and R₄, which may identical or different, represent a C₁-C₄ alkyl or a group of formula [(CH₂)ₘ—O]ₙ—H in which m and n are between 1 and 4; 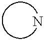
is a heterocycle containing 2 to 6 carbon atoms and at least one nitrogen atom.

5. The solution as claimed in claim 4, characterized in that the amine oxide is:
N-lauryldimethylamine oxide;
laurylpyridine oxide;
N-cetyldiethylamine oxide;
octylpyridine oxide;
N-myristylbis(2-hydroxyethyl)amine oxide.

6. The solution as claimed in claim 1, characterized in that the peroxidizing agent is:
peracetic acid, sodium peracetate, perpropionic acid, performic acid or potassium perpropionate;
magnesium monoperoxyphthalate hexahydrate (MPPM), perphthalic acid or perbenzoic acid;
sodium caroate or sodium percarbonate.

7. The solution as claimed in claim 1, characterized in that the anionic surfactant is chosen from (C₁₂-C₁₈)alkyl sulfates, (C₁₂-C₁₈)alkyl ether sulfates containing 2 to 4 mol of a (C₂-C₄)alkylene oxide; alkylsulfosuccinates, (C₈-C₁₆)alkylbenzensulfonates.

8. A composition for preparing a foaming decontaminating aqueous solution as claimed in claim 1.

9. The composition as claimed in claim 8, characterized in that it comprises, in separate packagings I and II:
the organic or mineral water-soluble peroxidizing agent and, optionally, a stabilizer for this water-soluble peroxidizing agent, in packaging I;
the other constituents in packaging II, and optionally all or some of the water, necessary for preparing the aqueous solution as claimed in claim 1.

10. The use of the aqueous solution as claimed in claim 1 for decontaminating dispersant systems and explosive agents liable to contain chemical or bacteriological toxic agents.

11. A process for decontaminating surfaces, characterized in that it consists in:
1) generating a foam from an aqueous solution as claimed in claim 1;
2) placing said foam in contact with the surface to be decontaminated for a few minutes;
3) recovering the effluents after liquefying the foam.

12. A process for destroying explosives liable to contain chemical or bacteriological toxic agents, characterized in that it consists in:
1) generating a foam from an aqueous solution as claimed in claim 1;
2) confining the explosives to be destroyed in a mobile chamber;
3) spraying the foam obtained in step 1) into said chamber;
4) igniting said explosives to be destroyed;
5) recovering the effluents after liquefying the foam.
